(19) United States
(12) Patent Application Publication
(10) Pub. No.: US 2026/0159867 A1
(43) Pub. Date: Jun. 11, 2026

(54) Preparation method and device for improving fermentation reaction efficiency of isomalto-oligosaccharide

(71) Applicant: Dahui International Food Co. Ltd, British Virgin Islands (GB)

(72) Inventor: Feng Gu, Bangkok (TH)

(21) Appl. No.: 19/254,058

(22) Filed: Jun. 30, 2025

(51) Int. Cl. C12P 19/04; C12M 1/00; C12M 1/06; C12N 9/26; C12P 19/14

(52) U.S. Cl. CPC C12P 19/04; C12M 27/02; C12M 29/08; C12M 47/10; C12N 9/2414; C12P 19/14; C12Y 302/01001; C12P 2201/00

(57) ABSTRACT

A preparation method for improving fermentation reaction efficiency of isomalto-oligosaccharide includes steps of: 1) mixing starch and water, then adding α-amylase and mixing thoroughly; 2) sending the starch slurry into a steam jet liquefier for liquefaction; 3) adding saccharification enzyme and waiting until the starch slurry is fully saccharified; 4) adding transglycosylation enzyme to the starch slurry to produce the isomalto-oligosaccharide; 5) adding active yeast for fermentation; 6) filtering the starch slurry to remove impurities; then adding activated charcoal for decolorization and deodorization; and 7) sending the starch slurry into a multi-effect evaporation concentrator to evaporate water in the starch slurry, thereby obtaining a final product. The method solves the problems of uneven reaction and long reaction time by feeding filtered air into a fermentation tank through aeration pipes. The filtered air can increase the oxygen content in the starch slurry and accelerate the reaction efficiency.

### BACKGROUND OF THE PRESENT INVENTION

### Field of Invention

The present invention relates to a technical field of isomalto-oligosaccharide preparation, and more particularly to a preparation method and a device for improving fermentation reaction efficiency of isomalto-oligosaccharide.

### Description of Related Arts

Isomalto-oligosaccharide (IMO), also known as branched oligosaccharide, is a functional oligosaccharide, belonging to a class of starch sugar. IMO is widely used in the fields of food and medicine because of its excellent physicochemical properties such as low sweetness, acid-resistance, heat-resistance, and inaccessibility to yeast. Conventionally, starch is commonly used as a raw material for industrially producing IMO by enzymatic method, but the IMO content is only about 50%. The high content of glucose and maltose will affect the functionality and physicochemical properties of IMO. Therefore, chromatographic column separation technology, ion exchange column separation technology, membrane filtration technology, and microbial fermentation technology are commonly used to remove glucose and maltose.

Chromatographic column separation technology, ion exchange column separation technology and membrane filtration technology are all cumbersome with low production efficiency and high cost, which restrict the development of IMO. Therefore, microbial fermentation technology is preferred in industrial production to remove glucose and maltose from IMO syrup.

In the production of IMO, conventional microbial fermentation technology has the following technical shortcomings: the stirring paddles in the reaction tank are not capable of uniformly stirring, which leads to a long fermentation time of the sugar solution in the whole tank. The fermentation time is usually more than 60 hours, resulting in low production efficiency, low equipment utilization, and high energy consumption. Moreover, a large number of by-products will be produced during the fermentation process, such as alcohol, which affects the texture and flavor of IMO product. The uneven stirring in the fermentation tank also results in an uneven reaction, affecting product quality. These problems increase the production costs, affect the product quality, and constrain the application of IMO. Therefore, a novel technical solution is needed to solve these problems.

### SUMMARY OF THE PRESENT INVENTION

An object of the present invention is to provide a preparation method for improving fermentation reaction efficiency of isomalto-oligosaccharide, so as to solve the technical problems of long fermentation time, low product purity, and by-products existing in conventional fermentation process by optimizing the fermentation process, thereby improving the product quality and production efficiency, making it more suitable for industrialized and large-scale production.

Another object of the present invention is to provide a device for improving fermentation reaction efficiency of isomalto-oligosaccharide, so as to to significantly shorten the fermentation time, improve IMO purity, and reduce the generation of by-products such as alcohol by modifying the equipment, thereby improving the product quality and production efficiency, making it more suitable for industrialized and large-scale production.

Accordingly, in order to accomplish the above objects, the present invention provides a preparation method for improving fermentation reaction efficiency of isomalto-oligosaccharide, comprising steps of:
1) mixing starch and water to form a starch slurry with a concentration of 30-40 wt. %, and adjusting a pH value of the starch slurry to 5.5-6.0, then adding α-amylase and mixing thoroughly;
2) sending the starch slurry into a steam jet liquefier for liquefaction at 108-110° C.; performing enzyme inactivation when a DE (dextrose equivalent) value is 20-25, and then cooling down the starch slurry;
3) sending the starch slurry into a saccharification tank with a temperature of 55-58° C., and adjusting the PH value of the starch slurry to 5.0-6.0; then adding saccharification enzyme and waiting until the starch slurry is fully saccharified;
4) adding transglycosylation enzyme to the starch slurry to produce the isomalto-oligosaccharide; after transglycosylation, inactivating the transglycosylation enzyme;
5) sending the starch slurry into a fermentation tank, adjusting the PH value of the starch slurry to 4.5-5.0 and keeping a temperature at 50-52° C.; then adding active yeast for fermentation, so as to consume glucose and maltose produced during transglycosylation; feeding filtered air into the fermentation tank through an air intake pipe arranged on one sidewall of the fermentation tank for aeration; after fermentation, inactivating the active yeast;
wherein aeration pipes are connected to the air intake pipe, and the aeration pipes are arranged at a bottom of the fermentation tank; multiple aeration ports are arranged on the aeration pipes; the filtered air is fed into the aeration pipes through the air intake pipe, and discharged through the aeration ports for aeration;
6) adding perlite to the starch slurry and mixing thoroughly; then filtering the starch slurry to remove impurities; then adding activated charcoal for decolorization and deodorization; and
7) adding activated charcoal to the starch slurry again for decolorization and deodorization, and then sending the starch slurry into a multi-effect evaporation concentrator to evaporate water in the starch slurry, thereby obtaining a final product.

Preferably, in the step 1), the starch is tapioca starch.

Preferably, in the step 5), a fermentation time is 20-30 hours.

Preferably, three rings of the aeration pipes are arranged at the bottom of the fermentation tank in a concentric form.

Preferably, 180 aeration ports are arranged in an outermost ring of the aeration pipes, 130 aeration ports are arranged in a middle ring of the aeration pipes, and 72 aeration ports are arranged in an innermost ring of the aeration pipes.

Preferably, openings of the aeration ports face downwards, avoiding reflux of the starch slurry.

Preferably, the step 5) further comprises using additional aeration pipes at a middle of the fermentation tank to improve aeration efficiency.

Preferably, the step 5) further comprises reducing foam generated during fermentation with a defoaming blade installed at a top of the fermentation tank, so as to prevent the starch slurry from overflowing the fermentation tank.

Preferably, the step 5) further comprises adding oxygen into the filtered air to further improve fermentation efficiency.

Preferably, in the step 6), the starch slurry is filtered with a rotary drum filter.

Preferably, the method further comprises a step 8) of processing the final product by spray drying.

The present invention also provides a device for improving fermentation reaction efficiency of isomalto-oligosaccharide, comprising:
a fermentation tank;
an air intake pipe arranged on one sidewall of the fermentation tank; and
aeration pipes connected to the air intake pipe, wherein the aeration pipes are arranged at a bottom of the fermentation tank; multiple aeration ports are arranged on the aeration pipes.

Preferably, three rings of the aeration pipes are arranged at the bottom of the fermentation tank in a concentric form.

Preferably, 180 aeration ports are arranged in an outermost ring of the aeration pipes, 130 aeration ports are arranged in a middle ring of the aeration pipes, and 72 aeration ports are arranged in an innermost ring of the aeration pipes.

Preferably, openings of the aeration ports face downwards, avoiding reflux of starch slurry.

Preferably, the device further comprises additional aeration pipes at a middle of the fermentation tank to improve aeration efficiency.

Preferably, the device further comprises a defoaming blade installed at a top of the fermentation tank to reduce foam generated during fermentation and prevent starch slurry from overflowing the fermentation tank.

The present invention solves the problems of uneven reaction and long reaction time in the prior art by feeding filtered air into the fermentation tank through the aeration pipes. The filtered air can increase the oxygen content in the starch slurry and accelerate the reaction efficiency between the yeast and the starch slurry, thereby shortening the fermentation time and improving the production efficiency. In addition, the present invention can also reduce by-products such as alcohol, reduce the bad flavor it brings to the IMO product, and improve the purity and quality of IMO, making it more suitable for industrialized and large-scale production. At the same time, the present invention adopts a defoaming blade to reduce foam production during fermentation, which is suitable for mass production.

These and other objectives, features, and advantages of the present invention will become apparent from the following detailed description, the accompanying drawings, and the appended claims.

### BRIEF DESCRIPTION OF THE DRAWINGS

FIG. 1 is a flow chart of a preparation method for improving fermentation reaction efficiency of isomalto-oligosaccharide according to an embodiment of the present invention;

FIG. 2 is a flow chart of the preparation method for improving fermentation reaction efficiency of isomalto-oligosaccharide according to another embodiment of the present invention;

FIG. 3 is a structural of a device for improving fermentation reaction efficiency of isomalto-oligosaccharide according to the present invention;

FIG. 4 is a top view of aeration pipes according to the present invention; and

FIG. 5 is a structural of the device for improving fermentation reaction efficiency of isomalto-oligosaccharide according to the present invention with additional aeration pipes.

### DETAILED DESCRIPTION OF THE PREFERRED EMBODIMENT

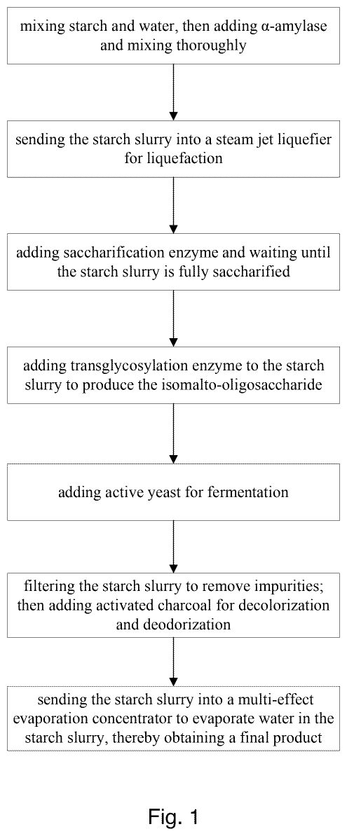

Referring to FIG. 1 of the drawings, a preparation method for improving fermentation reaction efficiency of isomalto-oligosaccharide is illustrated, comprising steps of:
1) mixing starch and water to form a starch slurry with a concentration of 30-40 wt. %, and adjusting a pH value of the starch slurry to 5.5-6.0, then adding α-amylase and mixing thoroughly;

2) sending the starch slurry into a steam jet liquefier for liquefaction at 108-110° C.; performing enzyme inactivation when a DE value is 20-25, and then cooling down the starch slurry;
3) sending the starch slurry into a saccharification tank with a temperature of 55-58° C., and adjusting the PH value of the starch slurry to 5.0-6.0; then adding saccharification enzyme and waiting until the starch slurry is fully saccharified;
4) adding transglycosylation enzyme to the starch slurry to produce the isomalto-oligosaccharide; after transglycosylation, inactivating the transglycosylation enzyme;
5) since the starch slurry after transglycosylation contains a large amount of glucose and maltose which affects the quality of IMO, sending the starch slurry into a fermentation tank, adjusting the PH value of the starch slurry to 4.5-5.0 and keeping a temperature at 50-52° C.; then adding active yeast for fermentation, so as to consume glucose and maltose produced during transglycosylation; wherein conventional fermentation stage is long lasting, usually more than 60 hours, so it is more important to reduce the reaction time in this stage; an air intake pipe is arranged on one sidewall of the fermentation tank, and aeration pipes connected to the air intake pipe is arranged at a bottom of the fermentation tank; three rings of the aeration pipes are arranged at the bottom of the fermentation tank in a concentric form; 180 aeration ports are arranged in an outermost ring of the aeration pipes, 130 aeration ports are arranged in a middle ring of the aeration pipes, and 72 aeration ports are arranged in an innermost ring of the aeration pipes; filtered air is fed into the aeration pipes through the air intake pipe for aeration; openings of the aeration ports face downwards, avoiding reflux of the starch slurry; during aeration, the filtered air provides a large amount of oxygen for microbial fermentation, accelerating a reaction speed while reducing the production of alcohol; at the same time, the filtered air forms bubbles in the starch slurry which moves from the bottom to a top of the fermentation tank, thereby stirring the starch slurry to improve the stirring efficiency, to accelerate the reaction efficiency of the yeast and the reaction liquid, and also to accelerate the evaporation of carbon dioxide and alcohol, which finally reduces residue of alcohol and other by-products in IMO, reduces the bad flavor brought by the by-products, and ensures a high efficiently; because the starch slurry keeps tumbling and generates a large amount of gas, a large amount of foam overflows the fermentation tank, so a defoaming blade is installed at the top of the tank to reduce foam generated during reaction and prevent the starch slurry from overflowing the fermentation tank; according to the present invention, a fermentation time is shortened from more than 60 hours to 20-30 hours, which greatly reduces the fermentation time; after fermentation, inactivating the active yeast;
6) adding perlite to the starch slurry and mixing thoroughly; then filtering the starch slurry with a rotary drum filter to remove impurities; then adding activated charcoal for decolorization and deodorization to keep the taste of the starch slurry; and
7) adding activated charcoal to the starch slurry again for decolorization and deodorization, and then sending the starch slurry into a multi-effect evaporation concentrator to evaporate water in the starch slurry, thereby obtaining a final product.

The step 5) further comprises adding oxygen into the filtered air to further improve fermentation efficiency.

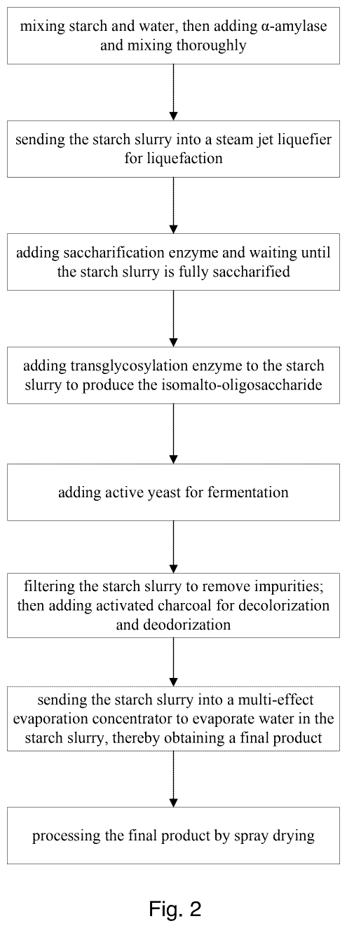

Referring to FIG. 2, the method further comprises a step 8) of processing the final product by spray drying.

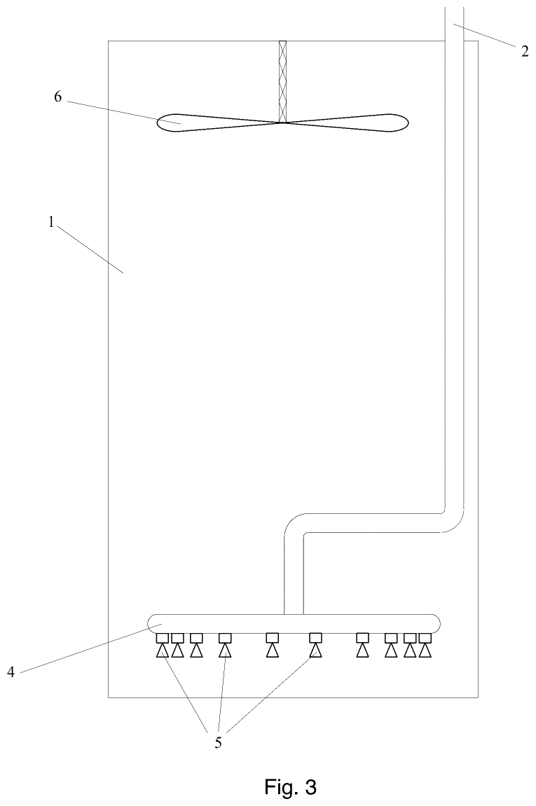

Referring to FIG. 3, a device for improving fermentation reaction efficiency of isomalto-oligosaccharide is illustrated, comprising:
a fermentation tank 1;
an air intake pipe 2 arranged on one sidewall of the fermentation tank 1;
aeration pipes 4 connected to the air intake pipe 1, wherein the aeration pipes 4 are arranged at a bottom of the fermentation tank 1; multiple aeration ports 5 are arranged on the aeration pipes 4; and
a defoaming blade 6 installed at a top of the fermentation tank 1 to reduce foam generated during fermentation and prevent starch slurry from overflowing the fermentation tank 1.

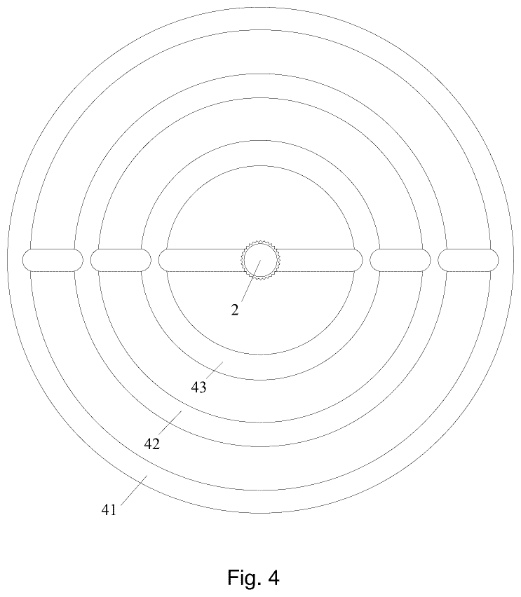

Referring to FIG. 4, three rings of the aeration pipes 4 are arranged at the bottom of the fermentation tank 1 in a concentric form, wherein 180 aeration ports 5 are arranged in an outermost ring 41 of the aeration pipes 4, 130 aeration ports 5 are arranged in a middle ring 42 of the aeration pipes 4, and 72 aeration ports 5 are arranged in an innermost ring 43 of the aeration pipes 4. Openings of the aeration ports 5 face downwards, avoiding reflux of starch slurry.

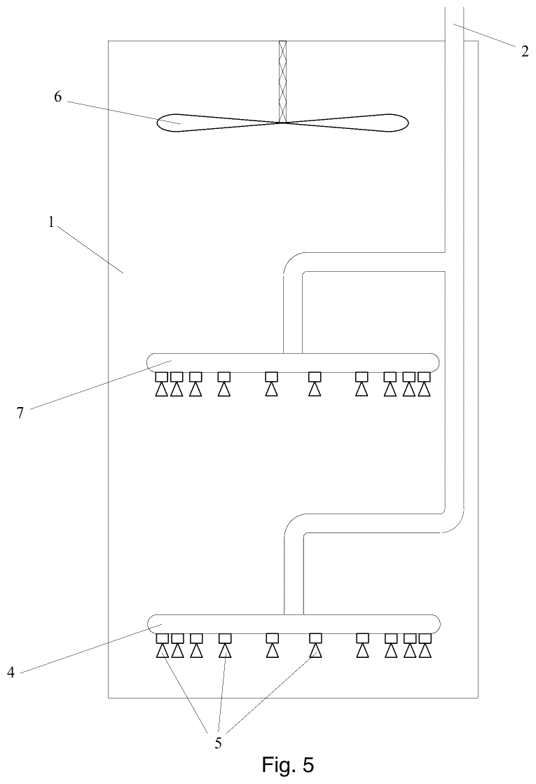

Referring to FIG. 5, the device further comprises additional aeration pipes 7 at a middle of the fermentation tank 1 to improve aeration efficiency.

### Preferred Embodiment 1

According to the preferred embodiment 1, a preparation method for improving fermentation reaction efficiency of isomalto-oligosaccharide comprises steps of:
1) mixing tapioca starch and water to form a starch slurry with a concentration of 35 wt. %, and adjusting a pH value of the starch slurry to 5.8, then adding α-amylase and mixing thoroughly;
2) sending the starch slurry into a steam jet liquefier for liquefaction at 109° C.; performing enzyme inactivation when a DE value is 22, and then cooling down the starch slurry;
3) sending the starch slurry into a saccharification tank with a temperature of 56° C., and adjusting the PH value of the starch slurry to 5.5; then adding saccharification enzyme and waiting until the starch slurry is fully saccharified;
4) adding transglycosylation enzyme to the starch slurry to produce the isomalto-oligosaccharide; after transglycosylation, inactivating the transglycosylation enzyme;
5) adjusting the PH value of the starch slurry to 4.8 and keeping a temperature at 51° C. ; then adding active yeast for fermentation; using aeration pipes to reduce a fermentation time to 30 hours, which is significantly more efficient than the 60 hours in the prior art; wherein during fermentation, filtered air is fed into the aeration pipes at a bottom of a fermentation tank through an air intake pipe for aeration; openings of the aeration ports face downwards, avoiding reflux of the starch slurry; during aeration, the filtered air forms bubbles in the starch slurry which moves from the bottom to a top of the fermentation tank, thereby stirring the starch slurry to improve the stirring efficiency, to accelerate the reaction efficiency of the yeast and the reaction liquid, and also to accelerate the evaporation of carbon dioxide and alcohol; a defoaming blade is installed at the top of the tank to reduce foam generated during reaction and prevent the starch slurry from overflowing the fermentation tank;
6) adding perlite to the starch slurry and mixing thoroughly; then filtering the starch slurry with a rotary drum filter to remove impurities; then adding activated charcoal for decolorization and deodorization to keep the taste of the starch slurry; and
7) adding activated charcoal to the starch slurry again for decolorization and deodorization, and then sending the starch slurry into a multi-effect evaporation concentrator to evaporate water in the starch slurry, thereby obtaining a final product.

### Preferred Embodiment 2

According to the preferred embodiment 2, a preparation method for improving fermentation reaction efficiency of isomalto-oligosaccharide comprises steps of:
1) mixing tapioca starch and water to form a starch slurry with a concentration of 30 wt. %, and adjusting a pH value of the starch slurry to 5.5, then adding α-amylase and mixing thoroughly;
2) sending the starch slurry into a steam jet liquefier for liquefaction at 108° C.; performing enzyme inactivation when a DE value is 20, and then cooling down the starch slurry;
3) sending the starch slurry into a saccharification tank with a temperature of 55° C., and adjusting the PH value of the starch slurry to 5.0; then adding saccharification enzyme and waiting until the starch slurry is fully saccharified;
4) adding transglycosylation enzyme to the starch slurry to produce the isomalto-oligosaccharide; after transglycosylation, inactivating the transglycosylation enzyme;
5) adjusting the PH value of the starch slurry to 4.5 and keeping a temperature at 50° C. ; then adding active yeast for fermentation; using aeration pipes to reduce a fermentation time to 30 hours, which is significantly more efficient than the 60 hours in the prior art; wherein during fermentation, filtered air is fed into the aeration pipes at a bottom of a fermentation tank through an air intake pipe for aeration; openings of the aeration ports face downwards, avoiding reflux of the starch slurry; during aeration, the filtered air forms bubbles in the starch slurry which moves from the bottom to a top of the fermentation tank, thereby stirring the starch slurry to improve the stirring efficiency, to accelerate the reaction efficiency of the yeast and the reaction liquid, and also to accelerate the evaporation of carbon dioxide and alcohol; a defoaming blade is installed at the top of the tank to reduce foam generated during reaction and prevent the starch slurry from overflowing the fermentation tank;
6) adding perlite to the starch slurry and mixing thoroughly; then filtering the starch slurry with a rotary drum filter to remove impurities; then adding activated charcoal for decolorization and deodorization to keep the taste of the starch slurry; and
7) adding activated charcoal to the starch slurry again for decolorization and deodorization, and then sending the starch slurry into a multi-effect evaporation concentrator to evaporate water in the starch slurry, thereby obtaining a final product.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A preparation method for improving fermentation reaction efficiency of isomalto-oligosaccharide, comprising steps of:
1) mixing starch and water to form a starch slurry with a concentration of 30-40 wt. %, and adjusting a pH value of the starch slurry to 5.5-6.0, then adding α-amylase and mixing thoroughly;
2) sending the starch slurry into a steam jet liquefier for liquefaction at 108-110° C.; performing enzyme inactivation when a DE (dextrose equivalent) value is 20-25, and then cooling down the starch slurry;
3) sending the starch slurry into a saccharification tank with a temperature of 55-58° C., and adjusting the PH value of the starch slurry to 5.0-6.0; then adding saccharification enzyme and waiting until the starch slurry is fully saccharified;
4) adding transglycosylation enzyme to the starch slurry to produce the isomalto-oligosaccharide; after transglycosylation, inactivating the transglycosylation enzyme;
5) sending the starch slurry into a fermentation tank, adjusting the PH value of the starch slurry to 4.5-5.0 and keeping a temperature at 50-52° C.; then adding active yeast for fermentation, so as to consume glucose and maltose produced during transglycosylation; to accelerate reaction and reduce effects of by-product comprising alcohol, feeding filtered air into the fermentation tank through an air intake pipe arranged on one sidewall of the fermentation tank for aeration; after fermentation, inactivating the active yeast;
wherein aeration pipes are connected to the air intake pipe, and the aeration pipes are arranged at a bottom of the fermentation tank; multiple aeration ports are arranged on the aeration pipes; the filtered air is fed into the aeration pipes through the air intake pipe, and discharged through the aeration ports for aeration;
6) adding perlite to the starch slurry and mixing thoroughly; then filtering the starch slurry to remove impurities; then adding activated charcoal for decolorization and deodorization; and
7) adding activated charcoal to the starch slurry again for decolorization and deodorization, and then sending the starch slurry into a multi-effect evaporation concentrator to evaporate water in the starch slurry, thereby obtaining a final product.

2. The method, as recited in claim 1, wherein in the step 1), the starch is tapioca starch.

3. The method, as recited in claim 1, wherein in the step 5), a fermentation time is 20-30 hours.

4. The method, as recited in claim 1, wherein three rings of the aeration pipes are arranged at the bottom of the fermentation tank in a concentric form.

5. The method, as recited in claim 4, wherein 180 aeration ports are arranged in an outermost ring of the aeration pipes, 130 aeration ports are arranged in a middle ring of the aeration pipes, and 72 aeration ports are arranged in an innermost ring of the aeration pipes.

6. The method, as recited in claim 1, wherein openings of the aeration ports face downwards, avoiding reflux of the starch slurry.

7. The method, as recited in claim 1, wherein the step 5) further comprises using additional aeration pipes at a middle of the fermentation tank to improve aeration efficiency.

8. The method, as recited in claim 1, wherein the step 5) further comprises reducing foam generated during fermentation with a defoaming blade installed at a top of the fermentation tank, so as to prevent the starch slurry from overflowing the fermentation tank.

9. The method, as recited in claim 1, wherein the step 5) further comprises adding oxygen into the filtered air to further improve fermentation efficiency.

10. The method, as recited in claim 1, wherein in the step 6), the starch slurry is filtered with a rotary drum filter.

11. The method, as recited in claim 1, further comprising a step 8) of processing the final product by spray drying.

12. A device for improving fermentation reaction efficiency of isomalto-oligosaccharide, comprising:
a fermentation tank;
an air intake pipe arranged on one sidewall of the fermentation tank; and
aeration pipes connected to the air intake pipe, wherein the aeration pipes are arranged at a bottom of the fermentation tank; multiple aeration ports are arranged on the aeration pipes.

13. The device, as recited in claim 11, wherein three rings of the aeration pipes are arranged at the bottom of the fermentation tank in a concentric form.

14. The device, as recited in claim 13, wherein 180 aeration ports are arranged in an outermost ring of the aeration pipes, 130 aeration ports are arranged in a middle ring of the aeration pipes, and 72 aeration ports are arranged in an innermost ring of the aeration pipes.

15. The device, as recited in claim 11, wherein openings of the aeration ports face downwards, avoiding reflux of starch slurry.

16. The device, as recited in claim 11, further comprising additional aeration pipes at a middle of the fermentation tank to improve aeration efficiency.

17. The device, as recited in claim 11, further comprising a defoaming blade installed at a top of the fermentation tank to reduce foam generated during fermentation and prevent starch slurry from overflowing the fermentation tank.
